(19) United States
(12) Patent Application Publication
(10) Pub. No.: US 2023/0257817 A1
(43) Pub. Date: Aug. 17, 2023

(54) IDENTIFICATION OF GENOME REGIONS ASSOCIATED WITH KIDNEY DISEASE AND TREATMENT

(71) Applicant: THE RESEARCH INSTITUTE AT NATIONWIDE CHILDREN'S HOSPITAL, Conlumbus, OH (US)

(72) Inventors: WILLIAM E. SMOYER, Bexley, OH (US); Sagar Bhayana, Conlumbus, OH (US); Shipra Agrawal, Upper Arlington, OH (US)

(21) Appl. No.: 18/016,153

(22) PCT Filed: Jul. 14, 2021
(86) PCT No.: PCT/US2021/041581

§ 371 Date: Jan. 13, 2023

Related U.S. Application Data
(60) Provisional application No. 63/051,565, filed on Jul. 14, 2020.

(51) Int. Cl. C12Q 1/6883; C12Q 1/6869

(52) U.S. Cl. CPC C12Q 1/6883; C12Q 1/6869; C12Q 2600/136; C12Q 2600/158

(57) ABSTRACT

Methods of identifying one or more genomic regions associated with kidney disease and/or its treatment are described. The methods include administering a glucocorticoid to a first group of subjects; administering a thiazolidinedione to a second group of subjects; and identifying a plurality of genomic regions affected in the first group and the second group, wherein the subjects have a kidney disease or are animal models of a kidney disease. Methods of identifying a drug for treatment of nephrotic syndrome are also described.

### CROSS-REFERENCE TO RELATED APPLICATIONS

The present application claims priority to U.S. Provisional Application No. 63/051,565, filed on Jul. 14, 2020, which is incorporated herein in its entirety.

### STATEMENT REGARDING FEDERALLY SPONSORED RESEARCH

This invention was made with government support under Grant No. DK110077 awarded by the National Institutes of Health. The Government has certain rights in this invention.

### BACKGROUND

Nephrotic Syndrome (NS) is defined as a group of glomerular diseases that are diagnostically characterized by massive proteinuria, edema, hypoalbimunemia and hyperlipidemia. Smoyer W E, Mundel P., J Mol Med (Berl)., 76(3-4):172-83 (1998). Three different variants of NS exist histologically minimal change disease (MCD), focal segmental glomerulosclerosis (FSGS) and membranous nephropathy (MN). Eddy A A, Symons J M., Lancet, 362(9384):629-39 (2003). NS is one of the common contributors of chronic kidney disease, responsible for 12% of kidney failure in adults and 20% in children. According to Center for Disease Control and prevention report, 50,633 deaths were reported in US in 2017 related to nephritis, nephrotic syndrome and nephrosis. Particularly with children, the annual incidence rate of NS reported is 2-7 cases per 100,000 children and prevalence of nearly 16 cases per 100,000. 90% of pediatric NS cases are idiopathic, but researchers have reported that some existing diseases and some specific genetic changes may be the cause of primary childhood NS.

The initial treatment of NS is high-dose daily glucocorticoids (GC), which unfortunately is often associated with significant side effects. While 80% of children achieve clinical remission (i.e., resolution of proteinuria and edema) after 4-6 weeks of GC therapy, but many children relapse after initial therapy, and as many as 50% of children develop frequently relapsing (FRNS) or steroid dependent NS (SDNS) Multiple relapses could lead to resistance to steroid and other available therapies. Such cases require either recurrent and/or prolonged GC therapy or alternative immunosuppressive medications which themselves are only partially effective and have significant toxicities. Moreover, ˜20% of children and ˜50% of adults present with or develop steroid resistant NS (SRNS), and are at the highest risk for progression to ESKD. MacHardy et al., Pediatr Nephrol., 24(11):2193-201 (2009). Therefore, there is an increasing demand for targeted therapies for the treatment of NS.

### SUMMARY

An attractive alternative to new drug development is to repurpose existing FDA-approved medications, which may markedly reduce costs and shorten the time required to gain regulatory approval. In this context, the inventors (and others) have reported the PPARy agonist, pioglitazone (Pio), as a potential alternative non-immunosuppressive treatment for NS. Agrawal et al., Mol Pharmacol 80, 389-399 (2011). Pio belongs to the thiazolinedione (TZD) class of drugs and is approved by the Food and Drug Administration (FDA) for the treatment of type II diabetes mellitus. Sarafidis et al., Am J Kidney Dis 55, 835-847 (2010). The inventors previously reported significant proteinuria reduction after Pio treatment in the puromycin aminonucleoside (PAN)-induced rat model of NS (PAN-NS), which was comparable to the proteinuria reduction achieved by GC treatment. Agrawal et al., Sci Rep 6, 24392 (2016). Since both GC and Pio activate nuclear receptors (NR3C1 AND PPARG, respectively), the inventors hypothesized that the similar proteinuria reducing effects of GC and Pio result from overlapping glomerular transcriptional patterns. To test this hypothesis, they compared the glomerular transcriptomes from rats with PAN-NS in whom proteinuria was reduced with either GC (immunosuppressive) or Pio (non-immunosuppressive) treatments. The rationale was that the identification of overlapping glomerular transcriptional targets induced by both GC and Pio would reveal common molecular pathways for proteinuria reduction in NS that could be exploited for future treatment of NS.

These studies demonstrated that although GC and Pio both reduced proteinuria in NS similarly, they did so by inducing both distinct and overlapping glomerular gene sets. Notably, informatics analyses of overlapping genes identified ECM proteins and lipid metabolism as novel targets for future therapies for NS, distinct from current immunosuppressive approaches.

### BRIEF DESCRIPTION OF THE FIGURES

FIGS. 1A-1E provide graphs showing Glucocorticoids and Pioglitazone Partially Reverse Nephrotic Syndrome-Associated Gene Expression Profile A: Urine protein-to-creatinine ratios (UPC) of individual animals on Day 11 of post-PAN, PAN+MP, PAN+Pio or PBS control; n=4 rats per group *P<0.05. B: Principal Component Analysis (PCA) plot representing unsupervised clustering of RNA-seq data. Each group is represented by a colored bubble with n=4 rats/group. The colored bubble represents the confidence interval of combined four points and each point within the bubble represents the transcriptome (16,915 annotated genes) from each rat's pooled glomeruli. C: Heat map representation of 1,872 differentially expressed genes (DEGs). Criteria for the selection: (1) DEGs were filtered based on FDR<0.05 in PAN vs. Control, (2) FDR>0.05 in PAN+MP vs. Control, and (3) At least 3 columns per gene showed normalized counts (NC)>25. Z-scores derived from normalized counts were used for heatmap construction, and centroid linkage hierarchical clustering was employed for heat map analysis. Red to blue scale denotes high Z-score to low Z-score. D: Venn diagram representing the number of genes that were either upregulated or downregulated in PAN vs. Control (FDR<0.05), and in treatment groups vs. PAN (FDR<0.05). BLUE color denotes downregulated genes and RED color denotes upregulated genes. FDR is defined as false discovery rate, adjusted for multiple testing with the Benjamini-Hochberg procedure. E: Line-plot representing the fold change (Log₂FC) of 319 and 126 DEGs significantly induced and suppressed by PAN respectively (FDR<0.05, PAN vs. Control) but significantly reversed by treatment groups (FDR<0.05, PAN+MP, PAN+Pio vs. PAN). BLACK line denotes PAN. RED line denotes PAN+MP. GREEN line denotes PAN+Pio. F: Bar-graph representing the functional annotation of 319 PAN-induced DEGs and 126 PAN-suppressed DEGs plotted based on enrichment scores using the Database for Annotation, Visualization, and Integrated discovery (DAVID) functional annotation analysis platform. Enrichment score ranks the biological significance of gene groups based on the overall Fisher exact score of all enriched annotation terms.

FIGS. 2A & 2B provide schematic representations of Drug-Nuclear Receptor-DEGs Interaction Network-Based Analysis of PAN-Induced and PAN-Suppressed DEGs Revealed 20 Genes-of-Interest (Method 1) A: An Ingenuity Pathway Analysis (IPA)-derived interaction network map of 319 PAN-induced and 126 PAN-suppressed genes with both nuclear receptors (Glucocorticoid receptor (NR3C1; shown in YELLOW) and PPARG receptor (PPARG; shown in BLUE)) and their respective agonists (Methylprednisolone; shown in BROWN; Dexamethasone, shown in PINK, and Pioglitazone; shown in GREEN). The interaction network map was generated via the curated IPA knowledge base. Genes in BOLD denote the 20 genes-of-interest that were commonly altered by both nuclear receptors (NR3C1 and PPARγ) and/or the receptors' agonists. B: The corresponding protein-protein interactions of the 20 genes-of-interest determined by Search Tool for the Retrieval of Interacting Genes/Proteins (STRING) platform. Four clearly segregated clusters became apparent based on their respective biological annotation (biological processes, molecular function, cellular compartment). GREY colored nodes represent proteins, and the line thickness between nodes signifies the strength of data supporting the association (i.e. thicker lines denote more available data sources showing the interaction, based on the STRING database). Interaction data sources included text mining, experiments, and databases. Minimum required interaction scores were set on medium confidence at 0.400. Clustering was performed using k-means clustering. Disconnected nodes were removed from the network.

FIGS. 3A-3C provide graphs and schematic representations showing GSEA enriched gene-sets encoding genes involved in remodeling of extracellular matrix and regulation of cell cycle (Method 2): Gene Set Enrichment Analysis (GSEA) show enrichment of NABA_MATRISOME_ASSOCIATED, NABA_ECM_REGULATORS, NABA_CORE_MATRISOME and SA_REG_CASCADE_OF_CYCLIN_EXPR related genes among the PAN, PAN+MP, and PAN+Pio genes that were ranked by normalized enrichment signal (NES) and q-value (FDR). The heat-map represents gene expression values of core-enriched genes. The core-enriched genes account for the enrichment signal and thus represent the small subset of all the genes that participate in a biological process. The GSEA was performed by the Bioconductor R package-cluster profiler using the Molecular Signatures Database (MSigDB) C2 curated gene set and Canonical Pathways (CP) as subcategory. Gene sets with a false discovery rate (FDR) value<0.05 after 1,000 permutations were considered to be significant. B: Ingenuity pathway analysis (IPA)-based interaction network formed between nuclear receptor (NR3C1, PPARG)—drug (dexamethasone, methylprednisolone, pioglitazone)—targets from the core-enriched genes from GSEA (shown in GREY color) (dexamethasone in PINK; methylprednisolone in BROWN; pioglitazone in GREEN) and the receptors (NR3C1, in YELLOW; PPARG, in BLUE). Genes in BOLD denote genes-of-interest that were shown to be commonly interacting with NR3C1 and PPARG receptors and/or their respective agonists. C: The corresponding protein-protein interactions of the 14 genes-of-interest determined by Search Tool for the Retrieval of Interacting Genes/Proteins (STRING) platform. Two clearly segregated clusters became apparent based on their respective biological annotation (biological processes, molecular function, cellular compartment). GREY colored nodes represent proteins and the line thickness between nodes signifies the strength of data supporting the association (i.e., thicker lines denote more available data sources showing the interaction, based on the STRING database). Interaction data sources included text mining, experiments and databases. Minimum required interaction scores were set on medium confidence at 0.400. Clustering was performed using k-means clustering. Disconnected nodes were removed from the network.

FIGS. 4A & 4B provide graphs showing the Correlation of Genes-Of-Interest with Rat and Human Nephrotic Syndrome-Associated Glomerular Gene Expression. Bar-Graph represents the qPCR validation of RNAseq data and filtration of 29 genes-of-interest into 17 genes-of-interest based on significance p<0.05 in expression between PAN and PAN+MP and PAN+Pio. The data is normalized to average of reference genes (RPL19 and PPIA) and is represented as Log₂(Fold Change) with control set at 0. The color-coded panels represent genes associated with biological processes. B: Heat-map represents the clinical correlation of qPCR validated genes in Hodgin focal segmental glomerulosclerosis (FSGS) RNAseq data using curated Nephroseq database. The table includes gene rank in FSGS vs. Normal condition and a fold change along with a p-value.

FIGS. 5A-5F provide graphs showing Glucocorticoids and Pioglitazone Both Reverse PAN-Induced mRNA Changes In Podocyte- and Mesangial Cell-Specific But Not Endothelial Cell Specific Gene Clusters: Heat-map representations (A, B and C) of treatment-induced gene expression changes in podocyte-specific genes (A: podocyte cluster, n=66 genes), mesangial cell-specific genes (B: mesangial cluster n=43 genes), and endothelial cell-specific genes (C: endothelial cluster n=42 genes). The heat map scale is based on raw z-scores calculated from the normalized read counts. The BLUE color denotes lower expression (i.e., downregulation), whereas the RED color denotes higher expression (i.e., upregulation). The normalized count cut-off for the selection of genes for each cluster was set as ≥500 D, E and F represents the PCA plots of respective heat maps with bubbles as confidence intervals of four points per group combined, where each point of a sample group denotes 66 genes for podocyte cluster, 43 genes for mesangial cluster and 42 genes for endothelial cluster.

FIG. 6 provides a graph showing In-Vitro Validation of Genes-Of-Interest in Podocytes and Mesangial Cells: (A-B) Bar graph represents mRNA expression of 17 genes-of-interest in PAN injured human podocyte cell line (MS13) (A) and human primary mesangial cells (B). Data is represented as a fold change and mRNA expression is normalized to mean of RPL19 and PPIA. Human Podocyte cell line were exposed to 2.5 μg/ml and 5 μg/ml of PAN whereas primary human mesangial cells were exposed to 5 μg/ml for 24 hrs and 48 hrs. N=2 samples per dose and per time-point. Unpaired t-test was applied and p<0.05 is considered statistically significant.

### DETAILED DESCRIPTION

The present invention provides methods of identifying one or more genomic regions associated with kidney disease and/or its treatment. The methods include administering a glucocorticoid to a first group of subjects; administering a thiazolidinedione to a second group of subjects; and identifying one or more genomic regions affected in the first group and the second group, wherein the subjects have a kidney disease or are animal models of a kidney disease. Another aspect of the invention provides methods of identifying a drug for treatment of nephrotic syndrome.

### Definitions

Unless otherwise defined, all technical and scientific terms used herein have the same meaning as commonly understood by one of ordinary skill in the art to which this invention pertains. In case of conflict, the present specification, including definitions, will control.

The terminology as set forth herein is for description of the embodiments only and should not be construed as limiting the application as a whole. Unless otherwise specified, “a,” “an,” “the,” and “at least one” are used interchangeably. Furthermore, as used in the description of the application and the appended claims, the singular forms “a”, “an”, and “the” are inclusive of their plural forms, unless contraindicated by the context surrounding such. Furthermore, the recitation of numerical ranges by endpoints includes all of the numbers subsumed within that range (e.g., 1 to 5 includes 1, 1.5, 2, 2.75, 3, 3.80, 4, 5, etc.).

Throughout this application, the term “about” is used to indicate that a value includes the standard deviation of error for the device or method being employed to determine the value, except that the value will never deviate by more than 5% from the value cited.

As used herein, the terms “treatment,” “treating,” and the like, refer to obtaining a desired pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

A “subject”, as used therein, can be a human or non-human animal Non-human animals include, for example, livestock and pets, such as ovine, bovine, porcine, canine, feline and murine mammals, as well as reptiles, birds and fish. Preferably, the subject is human Subjects can also be selected from different age groups. For example, the subject can be a child, adult, or elderly subject.

“Nucleic acid” or “oligonucleotide” or “polynucleotide”, as used herein, may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

The terms “identical” or percent “identity,” in the context of two or more polynucleotide sequences, refer to two or more sequences or subsequences that are the same or have a specified percentage of nucleotides that are the same e.g., 60% identity, preferably 65%, 70%, 75%, 80%, 85%, 90%, 91%, 92%, 93%, 94%, 95%, 96%, 97%, 98%, 99% or 100% identity over a specified region, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.0 algorithms and the default parameters discussed below are typically used.

Any “gene” is meant to refer to the polynucleotide sequence that encodes a protein, i.e., after transcription and translation of the gene a protein is expressed. As understood in the art, there are naturally occurring polymorphisms for many gene sequences. Genes that are naturally occurring allelic variations for the purposes of this invention are those genes encoded by the same genetic locus.

The inventors contemplate that genomic and/or transcriptomic changes observed in the kidney cells treated with glucocorticoid or thiazolidinedione alter cell signaling pathways of kidney cells (e.g., glomerular cells) such that the kidney cells show distinct intrinsic physiological characters. While identification of individual changes in transcript expression level may be associated with some physiological changes of the kidney cells and may be used as a marker to predict the status of the cell, such approach often fails to take account of individual variances in such changes or overall or net changes in the cell signaling networks that may lead to the similar or distinct physiological characteristics of the kidney cells. In particular, kidney cells treated with a glucocorticoid or thiazolidedione may show overlapping or commonly regulated targets that provide insight into kidney disease and potential therapies.

Identifying Genomic Regions Associated with Kidney Disease

In one aspect, the present invention provides a method of identifying one or more genomic regions associated with kidney disease and/or its treatment. The method includes administering a glucocorticoid to a first group of subjects; administering a thiazolidinedione to a second group of subjects; and identifying one or more genomic regions affected in the first group and the second group, wherein the subjects have a kidney disease or are animal models of a kidney disease. In some embodiments, a plurality of genomic regions are identified.

### Kidney Disease

Kidney disease, or renal disease, also known as nephropathy, is damage to or disease of a kidney. Nephritis is an inflammatory kidney disease and has several types according to the location of the inflammation. Nephrosis is non-inflammatory kidney disease. Nephritis and nephrosis can give rise to nephritic syndrome and nephrotic syndrome respectively. Kidney disease usually causes a loss of kidney function to some degree and can result in kidney failure, the complete loss of kidney function. Kidney disease includes both chronic and acute kidney disease. Chronic kidney disease is defined as prolonged kidney abnormalities (functional and/or structural in nature) that last for more than three weeks. Acute kidney disease is also known as acute kidney injury and is marked by the sudden reduction in kidney function over seven days. One type of chronic kidney disease is glomerular disease.

In some embodiments, the kidney disease is glomerular disease. Glomerular diseases affect the function of the kidneys and the glomeruli, which are small units within the kidney where blood is cleaned. Glomerular diseases include many conditions with a variety of genetic and environmental causes, but they fall into two major categories: Glomerulonephritis, which describes the inflammation of the membrane tissue in the kidney that serves as a filter, separating wastes and extra fluid from the blood, and glomerulosclerosis, which describes the scarring or hardening of the tiny blood vessels within the kidney. Although glomerulonephritis and glomerulosclerosis have different causes, they can both lead to kidney failure. Examples of glomerular disease include nephrotic syndrome, minimal change disease, diabetic nephropathy, and other conditions known to those skilled in the art.

Glomerular diseases damage the glomeruli, letting protein and sometimes red blood cells leak into the urine. Glomerular disease can also interfere with the clearance of waste products by the kidney, so they begin to build up in the blood. Furthermore, loss of blood proteins like albumin in the urine can result in a fall in their level in the bloodstream. When albumin leaks into the urine, the blood loses its capacity to absorb extra fluid from the body. Fluid can accumulate outside the circulatory system in the face, hands, feet, or ankles and cause swelling. Symptoms of glomerular disease include albuminuria, hematuria, reduced glomerular filtration rate, proteinuria, and edema.

In some embodiments, the glomerular disease is nephrotic syndrome. Nephrotic syndrome (NS) is a general term that refers to the loss of protein in the urine (proteinuria), hyperlipidemia (hypercholesterolemia and hypertriglyceridemia), and edema. Nephrotic syndrome involves changes in the pathology of cells in the kidney, such as podocytes. Many conditions are categorized as nephrotic syndromes, including minimal change disease (MCD), focal segmental glomerulosclerosis (FSGS), membranous nephropathy (MN) (also called membranous glomerulonephritis, MGN), and membranoproliferative glomerulonephritis (MPGN). For years pathologists found no changes in MCD tissue when viewing specimens under light microscopy, hence the name minimal change disease. With the advent of electron microscopy, the changes now known as the hallmarks for the disease include diffuse loss of podocyte foot processes, vacuolation of the podocyte foot processes, and growth of microvilli on the visceral epithelial cells. Diabetic nephropathy is the most common cause of nephrotic syndrome.

Model organisms are widely used to research human disease. This strategy is made possible by the common descent of all living organisms, and the conservation of numerous metabolic and developmental pathways. Animal models of a disease may have an existing, inbred or induced disease or injury that is similar to a human condition. Accordingly, in some embodiments, the subjects used in the method are animal models of kidney disease. A number of different animal models for kidney disease are known. See Hewitson et al., Methods Mol Biol., 466:41-57 (2009), the disclosure of which is incorporated herein by reference. In further embodiments, the subjects are animal models of glomerular disease, while in further embodiments the subjects are animal models of nephrotic syndrome. For example, rats injected with puromycin aminonucleoside are known to be a useful animal model of nephrotic syndrome.

The method includes the steps of administering a glucocorticoid to a first group of subjects and administering a thiazolidinedione to a second group of subjects. The subjects either have a kidney disease or are animal models of a kidney disease. The groups of subjects should include all of the same type of subject, and a sufficient number of individuals in order to provide statistically valid results. The groups of subjects include a plurality of individual subjects. In some embodiments, the groups include 2 to 5, 2 to 10, 5 to 10, 5 to 20, 10 to 20, 10 to 50, or 50 to 100 subjects.

### Glucocorticoids and Thiazolidinediones

The method of identifying genomic regions includes administering a glucocorticoid to a first group of subjects. Glucocorticoids are a recognized class of steroid-based drugs that bind to the glucocorticoid receptor, and include, for example, aldosterone, beclomethasone, betamethasone, budesonide, cloprednol, cortisone, cortivazol, eoxycortone, desonide, desoximetasone, difluorocortolone, luclorolone, flumethasone, flunisolide, fluocinolone, luocinonide, fluocortin butyl, fluorocortisone, fluorocortolone, fluorometholone, flurandrenolone, fluticasone, alcinonide, hydrocortisone, comethasone, meprednisone, methylprednisolone, mometasone, paramethasone, prednisolone, prednisone, tixocortol, triamcinolone, and others, and their respective pharmaceutically acceptable derivatives, such as beclomethasone diproprionate, dexamethasone 21-isonicotinate, fluticasone propionate, icomethasone enbutate, tixocortol 21-pivalate, triamcinolone acetonide, and others. In some embodiments, the glucocorticoid is selected from dexamethasone and methylprednisolone.

The method of identifying genomic regions also includes administering a thiazolidinedione to a second group of subjects. Thiazolidinediones are a class of heterocyclic compounds comprising a five-membered C3NS ring, and are typically used as insulin sensitizers. A variety of thiazolidinedione compounds are known to those skilled in the art. Examples of thiazolidinedione compounds include pioglitazone, ciglitazone, troglitazone, and rosiglitazone.

In some embodiments, the thiazolidinedione is pioglitazone. Pioglitazone is a is a peroxisome proliferator-activated receptor (PPAR)-α agonist, and has the structure shown in formula I below:

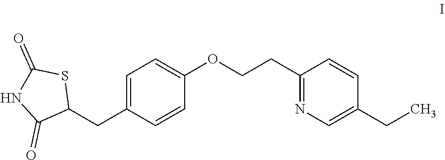 [SMILES: CCc1ccc(CCOc2ccc(CC3SC(=O)NC3=O)cc2)nc1]

The amount of the glucocorticoid and thiazolidinedione administered to the subject can be readily determined by one skilled in the art. In some embodiments, the amount of glucocorticoid and thiazolidinedione that are administered correspond to amounts known to be therapeutically effective, which is the amount of compound which will achieve the goal of decreasing disease severity while avoiding adverse side effects such as those typically associated with alternative therapies. However, in some embodiments, an amount which is merely effective to affect expression of genomic regions associated with kidney disease can be administered.

The glucocorticoid and thiazolidinedione can be administered together with a pharmaceutically acceptable carrier. A pharmaceutically acceptable carrier is one that does not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to a subject (e.g., human or animal). The use of such media and agents for pharmaceutically active substances is well known in the art.

### Genomic Regions

The present invention provides a method of identifying one or more genomic regions associated with kidney disease and/or its treatment. The methods identify variably-sized sets of residues in genomes (referred to herein as genomic regions) that are affected by a glucocorticoid and/or a thiazolidinedione. The genomic regions can include a range of base pairs. In some embodiments, the genomic region includes a number of base pairs ranging from 100 to 100,000, from 1000 to 100,000, from 5000 to 100,000, from 10,000 to 100,000, from 100 to 50,000, from 100 to 10,000, from 100 to 5,000, from 1000 to 50,000, or from 5,000 to 50,000. A genomic region can include genes and gene-sized polynucleotides.

The method includes detecting one or more, or a plurality, of different genomic regions associated with kidney disease and/or its treatment. In some embodiments, the method includes identifying from 2 to 1000 genomic regions, while in other embodiments the method includes identifying from 10 to 500, from 100 to 400, from 10 to 300, from 50 to 300, from 20 to 200, from 20 to 100, from 20 to 60, or from 20 to 40 genomic regions as regions of interest.

The method includes identifying genomic regions affected in both the first group and the second group. A genomic region is affected if its expression level is upregulator or downregulated, and the expression level of the identified genomic regions may be upregulated or downregulated. In some embodiments, the genomic regions that are upregulated or downregulated in both the first and second groups are identified. In some embodiments, the genomic regions that are upregulated in both the first and second groups are identified. In some embodiments, the genomic regions that are downregulated in both the first and second groups are identified. In further embodiments, the genomic regions affected are identified as being either upregulated or downregulated compared to subjects having untreated disease (i.e., subjects who have not been treated for kidney disease).

In some embodiments, the genomic regions may be those associated with the kidney, or kidney disease, and in particular the glomerular region of the kidney. Genomic regions associated with the kidney are known by those skilled in the art. Mahajan et al., Am J Hum Genet. 99(3), 636-646 (2016); Morris et al., Nat Commun. 10(1), 29, (2019). The glomerular region of the kidney depends on signaling between podocytes, endothelial cells, and mesangial cells. Accordingly, in further embodiments, each of the genomic regions identified is characterized as being podocyte-specific, endothelial cell-specific, or mesangial cell-specific. In other embodiments, the genes are associated with specific biochemical roles. For example, the genes may be associated with DNA damage and/or repair, transcription factors, the extracellular matrix, growth factors, lipid metabolism, or cytoskeletal rearrangements.

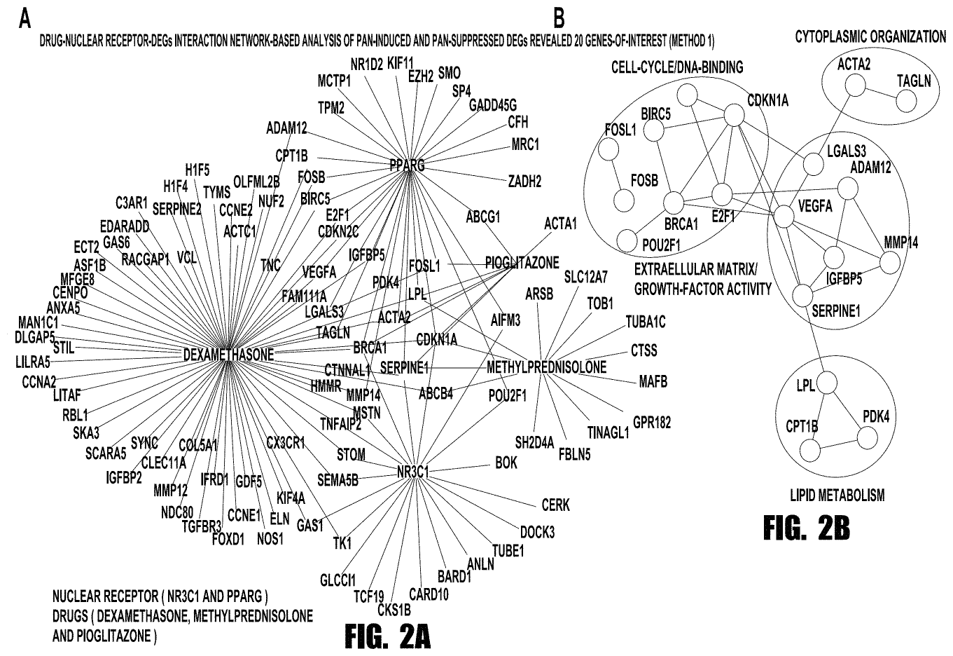

As shown in FIG. 2A, analysis may identify specific genes of interest. In some embodiments, the genomic regions are selected from the group of genes associated with functions consisting of cartinine palmitotransferase 1B (CPT1B), transgelin (TAGLN), pyruvate dehydrogenase kinase 4 (PDK4), cyclic dependent kinase inhibitor 1B (CDKN1B), cyclic dependent kinase inhibitor 1A (CDKN1A), cyclin-dependent kinase inhibitor 2C (CDKN2C), baculoviral IAP repeat containing 5 (BIRC5), serpin family E member 1 (SERPINE1), E2F transcription factor 1 (E2F1), ADAM metallopeptidase domain 12 (ADAM12), BRCA1 DNA repair associated (BRCA1), FosB proto-oncogene (FOSB), AP-1 transcription factor subunit, fos-like antigen 1 (FOSL1), actin gamma 2 (ACTA2), lipoprotein lipase (LPL), matrix metalloproteinase14 (MMP14), matrix metalloproteinase 2 (MMP2), apoptosis inducing factor mitochondria associated 3 (AIFM3), vascular endothelial growth factor A (VEGFA), and insulin-like growth factor binding protein 5 (IGFBP5), collagen, type 1, alpha 1 (COL1A1), lectin galactoside-binding soluble 3 (LGALS3) angiopoietin-like 4 (ANGPTL4), POU class2 homeobox 1 (POU2F1), leukemia inhibitory factor (LIF), fibroblast growth factor 1 (FGF1), chemokine (c-c motif) ligand 2 (CCL2), cyclin-dependent kinase 4 (CDK4) and transforming growth factor b2 (TGFB2).

The genomic regions are identified using transcriptomic analysis. The transcriptome is the set of all RNA transcripts (associated with the process of transcript production), including coding and non-coding, in a subject or a population of cells. One or more subtypes of transcriptomics data can be used to identify a genomic region. Exemplary transcriptomics data includes, but not limited to, expression levels of a plurality of mRNAs as measured by quantities of the mRNAs, maturation levels of mRNAs (e.g., existence of poly A tail, etc.), and/or splicing variants of the transcripts. The selection of genes and/or the number of genes to determine molecular signature related to kidney disease may differ, or minimally overlap with the selection of genes and/or the number of genes to determine molecular signature related to sensitivity to various types of treatment. However, the genes to be included in the relevant transcriptomics data set may include genes not associated with a disease (e.g., housekeeping genes), including, but not limited to, those related to transcription factors, RNA splicing, tRNA synthetases, RNA binding protein, ribosomal proteins, or mitochondrial proteins, or noncoding RNA (e.g., microRNA, small interfering RNA, long non-coding RNA (lncRNA), etc.).

The identification of genomic regions can be performed on RNA isolated from the kidney of the subjects. In some embodiments, identification of genomic regions (e.g., by RNA sequencing) is performed on RNA isolated from the kidney glomeruli of the subjects.

Any suitable methods of obtaining a kidney sample (e.g., kidney glomeruli) from the subjects (or healthy tissue from a patient or a healthy individual as a comparison) are contemplated. Most typically, a kidney sample can be obtained from the patient via a biopsy (including liquid biopsy, or obtained via tissue excision during a surgery or an independent biopsy procedure, etc.), which can be fresh or processed (e.g., frozen, etc.) until further process for obtaining omics data from the tissue.

RNA (e.g., mRNA, miRNA, siRNA, shRNA, etc.) can be obtained from kidney cells, isolated, and further analyzed to obtain transcriptomic data. Alternatively and/or additionally, a step of obtaining genomics data may include receiving data from a database that stores transcriptomic information of one or more subjects. For example, transcriptomics data may be obtained from isolated RNA from the subject's kidney tissue, and the obtained data may be stored in a database (e.g., cloud database, a server, etc.) with other transcriptomic data obtained from other subjects.

In some embodiments, the transcriptome data set includes sequence information and expression level (including expression profiling or splice variant analysis) of RNA(s) (preferably cellular mRNAs) that is obtained from the first and second groups of subjects. There are numerous methods of transcriptomic analysis known in the art, and all of the known methods are suitable for use in the methods described herein (e.g., RNAseq, Next Generation Sequencing, RNA hybridization arrays, qPCR, etc.). See Hrdlickova et al., Wiley Interdiscip Rev RNA, 8(1):10.1002/wrna.1364 (2017), and Conesa et al., Genome Biol., 17:13 (2016), the disclosures of which are incorporated by reference herein. Preferred materials include mRNA and primary transcripts (hnRNA), and RNA sequence information may be obtained from reverse transcribed polyA⁺-RNA, which is in turn obtained from a tumor sample and a matched normal (healthy) sample of the same patient. It should be noted that while polyA⁺-RNA is typically preferred as a representation of the transcriptome, other forms of RNA (hn-RNA, non-polyadenylated RNA, siRNA, miRNA, etc.) are also suitable for use herein. Preferred methods include quantitative RNA (hnRNA or mRNA) analysis and/or RNA sequencing (RNAseq). In other aspects, RNA quantification and sequencing is performed using RNA-seq, qPCR and/or rtPCR based methods, although various alternative methods (e.g., solid phase hybridization-based methods) are also suitable.

### Pathway Analysis

Without wishing to be bound by any specific theory, the inventors contemplate that the RNA expression profiles of the kidney tissue are correlated with the genes and intracellular signaling networks that are relevant to kidney disease and/or its treatment. Thus, the RNA expression profiles of the kidney tissue obtained from subjects of group one and group two can be integrated into a pathway model to generate information on suitable targets for treatment of kidney disease.

To computationally identify genomic regions associated with kidney disease and/or its treatment, various embodiments of the invention include functional gene annotation tools. For example, Ingenuity Pathway Analysis software can be used to identify the genomic regions associated with kidney disease and/or its treatment. Other useful software for functional gene analysis includes DAVID Bionformatics resources, STRING functional protein association networks, and Gene Set Enrichment Analysis. Accordingly, in some embodiments, the method further comprises analysis of the identified genomic regions (i.e., the target-drug interaction network) using Ingenuity pathway analysis of the identified genomic regions. In further embodiments, the ingenuity pathway analysis characterizes the genomic regions as being involved in the genes selected from the group consisting of genes relating to the extracellular matrix, core-matrisome, cell-cycle, DNA damage-repair, lipid metabolism, growth factors, cytokine activity, cell proliferation, and cell membrane glycoprotein levels.

In some embodiments, the method further comprises characterizing the genomic regions identified using functional annotation cluster analysis. Characterizing the genomic regions includes determining the function of the genomic region, including determining whether the genomic region is expressed as a protein, is a promoter or other control element, and what biochemical function it is associated with. In further embodiments, the functional annotation cluster analysis characterizes the genomic regions as being involved in the genes selected from the group consisting of genes relating to the extracellular matrix, core-matrisome, cell-cycle, DNA damage-repair, lipid metabolism, growth factors, cytokine activity, cell proliferation, and cell membrane glycoprotein levels.

### Methods for Identifying a Drug

Another aspect of the invention provides a method of identifying a drug for treatment of nephrotic syndrome. The method includes administering a glucocorticoid to a first group of subjects; administering thiazolidinedione to a second group of subjects; identifying a plurality of genomic regions affected in the first group and the second group, and identifying a drug for treatment of nephrotic syndrome if it is known to affect a genomic region affected in the first group and/or the second group of subjects; wherein the subjects have a kidney disease or are animal models of a kidney disease.

The method includes the step of identifying a drug for treatment of nephrotic syndrome if it is known to affect a genomic region affected in the first group and/or the second group of subjects. For example, depending on the genomic regions identified, the drugs may be known to affect DNA damage and/or repair, transcription factors, the extracellular matrix, growth factors, lipid metabolism, or cytoskeletal rearrangements. In further embodiments, the drugs may be known to affect cartinine palmitotransferase 1B (CPT1B), transgelin (TAGLN), pyruvate dehydrogenase kinase 4 (PDK4), cyclic dependent kinase inhibitor 1B (CDKN1B), cyclic dependent kinase inhibitor 1A (CDKN1A), cyclin-dependent kinase inhibitor 2C (CDKN2C), baculoviral IAP repeat containing 5 (BIRC5), serpin family E member 1 (SERPINE1), E2F transcription factor 1 (E2F1), ADAM metallopeptidase domain 12 (ADAM12), BRCA1 DNA repair associated (BRCA1), FosB proto-oncogene (FOSB), AP-1 transcription factor subunit, fos-like antigen 1 (FOSL1), actin gamma 2 (ACTA2), lipoprotein lipase (LPL), matrix metalloproteinase14 (MMP14), matrix metalloproteinase 2 (MMP2), apoptosis inducing factor mitochondria associated 3 (AIFM3), vascular endothelial growth factor A (VEGFA), and insulin-like growth factor binding protein 5 (IGFBP5), collagen, type 1, alpha 1 (COL1A1), lectin galactoside-binding soluble 3 (LGALS3) angiopoietin-like 4 (ANGPTL4), POU class2 homeobox 1 (POU2F1), leukemia inhibitory factor (LIF), fibroblast growth factor 1 (FGF1), chemokine (c-c motif) ligand 2 (CCL2), cyclin-dependent kinase 4 (CDK4) and transforming growth factor b2 (TGFB2).

The present invention is illustrated by the following example. It is to be understood that the particular example, materials, amounts, and procedures are to be interpreted broadly in accordance with the scope and spirit of the invention as set forth herein.

### Example

### Glomerular Transcriptomic Analysis of Glucocorticoid- and Pioglitazone-Treated Nephrotic Syndrome

The inventors utilized strategy of repurposing drugs as it is an attractive proposition because of low costs and shorter development times. Their lab and others have initially reported pioglitazone as an alternate therapy for NS. Agrawal et al., Mol Pharmacol., 80(3):389-99 (2011) Pioglitazone belongs to the thiazolidinedione (TZD) class of drugs, approved by FDA for treatment of type II diabetes mellitus. Sarafidis et al., Am J Kidney Dis., 55(5):835-47 (2010). A significant reduction in proteinuria in PAN-treated rats on using this drug was reported, exhibiting comparable efficacy when compared to GC treatment. Agrawal et al., Sci Rep., 6:24392 (2016). Notably, pioglitazone, a PPARy agonist and methylprednisolone, a NR3C1 agonist, both nuclear receptor ligands, act similarly via nuclear receptor signaling cross talk. The question arises is whether pioglitazone activates same class of molecules as glucocorticoids to have a similar effect, and/or if common signaling downstream targets could be identified for development of effective therapeutic options. All these questions and concerns led the inventors to design a study where they compared the transcriptomic profiles of glomeruli from methylprednisolone (NR3C1 agonist) and pioglitazone (PPARy agonist) treated rats by performing RNA sequencing. The usefulness of this resource not only led them to screen for common pathways or targets or transcriptomic features but also the specific cell types affected in glomeruli and its cellular dynamics in detail.

### Material and Methods

### Animal Study Design

Proteinuria was induced in male Wister rats (body weight˜′50 g, age˜45-50 d) by single tail vein injection of Puromycin aminonucleoside (PAN, #P7130, Sigma; St. Louis, Mo.) −50 mg/kg, n=4 on Day 0, while the control group received saline injection (n=4). PAN-induced proteinuria was treated daily with methylprednisolone (15 mg/kg, n=4, Solu-Medrol; Pfizer Inc., New York, N.Y.) via intraperitoneal injection or with pioglitazone (10 mg/kg, Actos; Takeda, Deerfield, Ill.) via oral gavage until day 11 of post PAN-injection. Morning spot urine samples were collected on Day 0 (before PAN injection) and day 11 for urinary protein: creatinine ratio (UPC) analysis. Kidneys were harvested at day 11 and were processed for glomerular isolation.

### UPC Measurement

UPC was measured by Antech Diagnostics (Morrisville, N.C.) using standard techniques that are fully compliant with Good Laboratory practice regulations. See Agrawal et al., Sci Rep 6, 24392 (2016)

### Glomerular Isolation

Glomeruli were isolated using sieving method where kidney cortex was pared using curved scissors into the petri lid with cold PBS, minced well, drained and washed onto pre-moistened No. 80 sieve. Using squeeze bottle, minced kidneys on No. 80 sieve were washed thoroughly and transferred to No. 140 sieve. No. 140 and 200 are stacked together to catch smaller material. Kidney material was then ground through No. 140 sieve with very gentle pressure using bottom of cold round 250 ml beaker. The ground kidney was washed back and forth well over sieve No. 140 onto No. 200. This catch material is the glomeruli. The glomeruli were washed with cold PBS to get rid of any contaminants and the final washed material was collected into labeled 50 ml conical tube. The tube was spun at ˜1500 rpm for 3 min Glomeruli precipitate was then suspended in RLT buffer (Qiagen, Germantown, Md.) containing β-mercaptoethanol (#M6250 Sigma).

### Total RNA Isolation and DNA Digestion

Total RNA from the isolated glomeruli was isolated using the RNeasy mini Kit (Qiagen) according to the manufacturer's instructions. The purity and yield of RNA was determined by measuring the absorbance at 230, 260 and 280 nm. Briefly, 1 ug of RNA was subjected to DNase (Ambion, Thermo Fisher Scientific) digestion at 37° C. for 30 min followed by DNase inactivation with 5 mM EDTA at 75° C. for 10 min.

### RNA Sequencing Library Preparation and Sequencing

RNA quality was assessed using the Agilent 2100 Bioanalyzer and RNA Nano Chip Kit (Agilent Technologies, CA) to ensure that the RNA Integrity Number (RIN) was ≥7. RNA-seq libraries were then generated using TruSeq Standard total RNA with Ribo-Zero Globin Complete kit (Illumina, CA). Briefly, ribosomal RNA (rRNA) was removed from 350 ng of total RNA with biotinylated, target-specific oligos combined with Ribo-Zero rRNA removal beads from the Human/Mouse/Rat Globin kit. To generate directional signals in RNA seq data, libraries were constructed from first strand cDNA using ScriptSeq™ v2 RNA-Seq library preparation kit (Epicentre Biotechnologies, WI). Briefly, 50 ng of rRNA-depleted RNA was fragmented and reverse transcribed using random primers containing a 5′ tagging sequence, followed by 3′ end tagging with a terminal-tagging oligo to yield di-tagged, single-stranded cDNA. Following purification using a magnetic bead-based approach, the di-tagged cDNA was amplified by limit-cycle PCR using primer pairs that anneal to tagging sequences and add adaptor sequences required for sequencing cluster generation. Amplified RNA-seq libraries were purified using AMPure XP System (Beckman Coulter). The quality of libraries was determined via Agilent 2200 TapeStation using High Sensitivity D1000 tape, and quantified using Kappa SYBR®Fast qPCR kit (KAPA Biosystems, Inc, MA). Approximately, 90-125 million paired-end 150 bp reads were generated per sample using the Illumina HiSeq4000 platform. Raw data were converted to FASTQ using Illumina's bc12fastq application. Sequencing adapters matching at least 6 bases were then removed from the reads, as well as low-quality bases (<10) using v1.10 of cutadapt. An alignment report was also generated, using custom scripts, and manually reviewed to ensure that at least ˜80% of reads aligned to the expected reference, and that at least ˜50% of the reads aligned to features annotated as protein coding.

### RNA-Seq Data Analysis

Each sample was aligned to the Rnor_6.0 assembly of the Rattus norvegicus reference from the National Center for Biotechnology Information (NCBI) using version of 2.5.0c of the RNA-Seq aligner STAR, as described at the Oxford Academic Bioinformatics website. Transcript features were identified from the general feature format (GFF) file that came with the assembly from the NCBI. Feature coverage counts were calculated using HTSeq, using the instructions provided at the HTSeq website for “Analysing high-throughput sequencing data with Python.” The raw RNA-Seq gene expression data was normalized, and post-alignment statistical analyses and figure generation were performed using DESeq2 (Love et al., “Moderated estimation of fold change and dispersion for RNA-seq data with DESeq2,” Genome Biology volume 15, Article number: 550 (2014)) and custom analysis scripts written in R. Comparisons of gene expression and associated statistical analyses were made between different conditions of interest using the normalized read counts. All fold change values are expressed as test condition/control condition, where values less than one are denoted as the negative of its inverse (note that there will be no fold change values between −1 and 1, and that the fold changes of “1” and “−1” represent the same value). Transcripts were considered significantly differentially expressed using a 10% false discovery rate (DESeq2 adjusted p value<=0.1). Genes were removed from comparisons if they were not expressed above a background threshold (0.5 reads per million) for most samples within each group.

### Functional Gene Annotation Tools

Functional gene analysis was performed by Ingenuity Pathway Analysis software (IPA), DAVID Bioinformatics Resources 6.8, NIAID/NIH, STRING functional protein association networks and Gene Set Enrichment Analysis (GSEA). The inventors utilized IPA for building drug-target interaction network by exploring the connections between nuclear receptors, their agonists and the streamlined genes data using IPA knowledgebase as the reference. They also used IPA's disease view and toxicity feature to link experimental data to understand biological functional and pharmacological response.

DAVID functional annotation tool aids in unraveling biological processes associated with a gene-lists using gene co-occurrence probability. DAVID 6.8 contains information on over 1.5 million genes from more than 65,000 species. STRING database entails known and predicted protein-protein interactions. These interactions stem from computational prediction, from knowledge transfer between organisms, and from interactions aggregated from other (primary) databases. The STRING database currently covers 24,584,628 proteins and 5,090 organisms. GSEA identifies biological pathways that are enriched in a gene list more than would be expected by chance. GSEA progressively examines genes from the top to the bottom of the ranked list, increasing the enrichment score (ES) if a gene is part of the pathway and decreasing the score otherwise. The ES score is calculated as the maximum value of the running sum and normalized relative to the pathway size, resulting in a normalized enrichment score (NES) that reflects the enrichment of the pathway in the list. Positive and negative NES values represent enrichment at the top and bottom of the list, respectively. GSEA uses C2 curated gene sets collection in molecular signatures database (MSigDb).

### Cell Culture

The conditionally immortalized human podocyte cell line (MS13) and human primary mesangial cells were cultured as previously described. Sharma et al., J Am Soc Nephrol 28, 2618-2630 (2017). 3×10⁴ undifferentiated podocytes were plated on a 6-well plate and were incubated at 37° C. for 12-14 days prior to the start of experiment whereas 1×10⁵ mesangial cells were plated on a 6-well plate a day prior to start of experiment. Further, podocytes and mesangial cells were serum starved 0/N with 1% FCS containing RPMI media, before the experiment. The following day, podocytes were exposed to 2.5 and 5 μg/ml of PAN for 24 and 48 hrs whereas in case of mesangial cells, 5 μg/ml of PAN (Sigma; St. Louis, Mo.) was added for 24 and 48 hrs. Cells were lysed in 400 μl of RNA lysis buffer provided by Ambion (#AM1560).

Total RNA Isolation from Cultured Cells

Total RNA from the cultured podocytes and mesangial cells was isolated using the miRVANA miRNA isolation kit (Ambion, ThermoScientific) according to the manufacturer's instructions. The RNA was eluted in 40 μl of nuclease free water. The purity and yield of RNA was determined by measuring the absorbance at 230, 260 and 280 nm. Briefly, 1 μg of RNA was subjected to DNase (Ambion, Thermo Fisher Scientific) digestion at 37° C. for 30 min followed by DNase inactivation with 5 mM EDTA at 75° C. for 10 min.

cDNA Synthesis and Real-Time PCR

250 μg of total isolated RNA was reverse transcribed to cDNA by iScript cDNA synthesis kit according to manufacturer's instructions (#1708890. Bio-Rad). The resulting cDNA was diluted to 1:5 with nuclease free water. Diluted cDNA was finally used for relative mRNA quantitation of 17 targets-of-interest using SYBER green based Bio-Rad CFX96 Real-Time PCR machine (Bio-Rad Labs, Hercules, Calif.). Both RPL19 and PPIA were used as reference genes.

### Results

### Glucocorticoids and Pioglitazone Both Partially Reverse Nephrotic Syndrome-Associated Glomerular Gene Expression Abnormalities

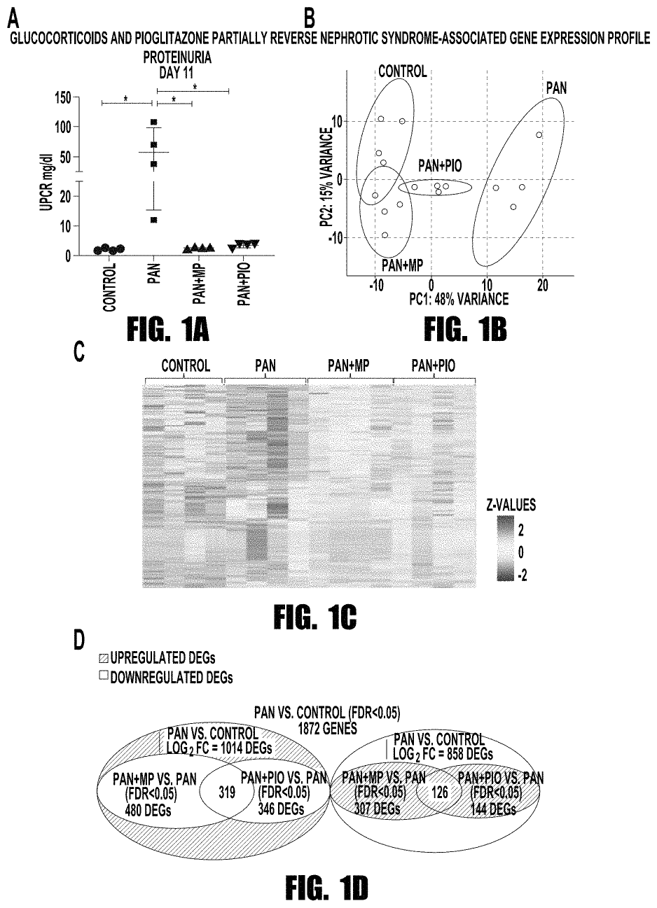

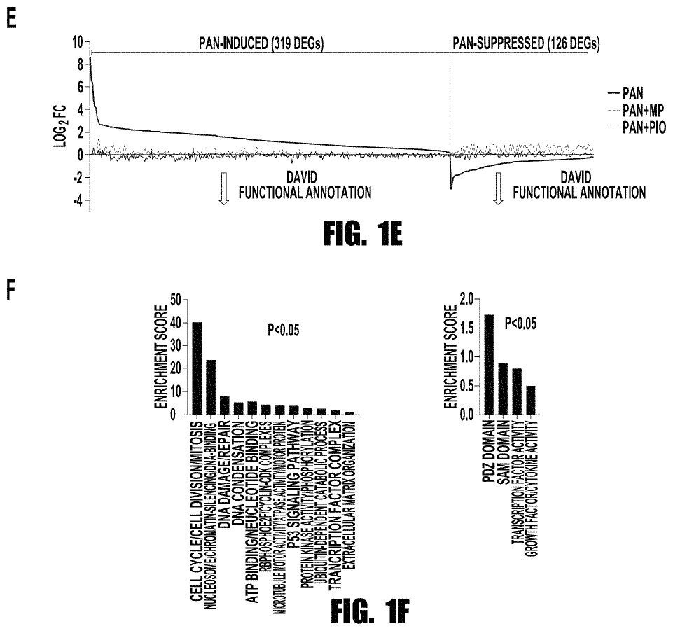

The inventors previously demonstrated that both GC (i.e. methylprednisolone; MP) and Pio significantly reduced proteinuria in rats with PAN-induced NS. Agrawal et al., Sci Rep 6, 24392 (2016). To attempt to identify common molecular targets/pathways of proteinuria reduction, RNA sequencing was performed on glomeruli isolated from 4 rats from each of 4 experimental groups: 1) healthy controls, 2) PAN+sham treatment, 3) PAN+GC, and 4) PAN+Pio. As expected, PAN treatment induced significant proteinuria (Urine protein to creatinine ratio (UPCR)=57.2±41.6 mg/mg vs. 2.03±0.42 mg/mg in healthy controls; * P<0.05) at day 11 following a single PAN dose (50 mg/kg IV) (FIG. 1A). Treatment with either MP [2.64±0.32 mg/mg (95% reduction); * P<0.05] or Pio [3.45±0.82 mg/mg (94% reduction); * P<0.05] significantly reduced proteinuria. Subsequent unsupervised clustering of ˜17,000 glomerular genes using PCA was used to define and compare the glomerular transcriptomes from each rat from each group, which revealed discrete transcriptional profiles for each treatment group (FIG. 1B). Importantly, the PAN-induced NS glomerular transcriptome was entirely distinct and non-overlapping with the healthy control glomerular transcriptome. In contrast, the glomerular transcriptomic profiles of the PAN+MP and PAN+Pio groups both revealed substantial transcriptomic profile shifts toward healthy controls. Notably, however, despite similar reductions in proteinuria the MP and Pio transcriptomic profiles had only minimal overlap of their 95% confidence intervals (i.e. colored spheres), and the MP profile had only modest overlap with the healthy control profile (FIG. 1B). These transcriptomic profiles strongly suggested that both MP and Pio reduce proteinuria by partially restoring a healthy glomerular gene expression profile.

Since both MP and Pio significantly ameliorated proteinuria, analysis was focused on the similarities between these transcriptomic profiles. The expression pattern of genes in individual samples was reaffirmed by filtering genes based on strict selection criteria, which included: 1) Gene expression levels significantly differed (False discovery rate (FDR)<0.05) between PAN vs. controls, 2) Similar or comparable expression levels between PAN+MP and controls (FDR>0.05), 3) Normalized read counts (NC)>25 in at least 3 samples in either group, and 4) Genes that were well characterized and map to software platforms developed for Gene Ontology (GO) enrichment and pathway analysis. Using these selection criteria, we identified 1,872 differentially expressed genes (DEGs), which are plotted as a heat map in FIG. 1C. A Venn diagram shown in FIG. 1D summarizes the distribution of these 1,872 DEGs that were specifically up- or down-regulated during PAN-NS, as well as following treatment with either MP or Pio. Specifically, PAN-NS glomeruli had 1,014 significantly upregulated and 858 significantly downregulated genes compared to healthy controls, which identified a distinct pattern of glomerular transcriptional dysregulation associated with induction of significant proteinuria in this NS model. In response to proteinuria reduction, MP significantly ameliorated 480 (47%) and Pio significantly ameliorated 346 (34%) of the 1,014 glomerular genes upregulated in PAN-NS (FDR<0.05). Similarly, MP significantly ameliorated 307 (36%) and Pio significantly ameliorated 144 (17%) of the 858 glomerular genes downregulated in PAN-NS (FDR<0.05). Through overlap analysis, we identified 319 downregulated DEGs (genes significantly induced by PAN but ameliorated by both MP and Pio) and 126 upregulated DEGs (genes significantly suppressed by PAN but ameliorated by both MP and Pio) that were common to both MP (immunosuppressive) and Pio (non-immunosuppressive) proteinuria reduction treatments, and categorized these as PAN-induced and PAN-suppressed DEGs, respectively (FIG. 1E). This approach identified these two groups of DEGs as potential common molecular regulators of proteinuria reduction, and thus highlighted their potential as novel future therapeutic targets for proteinuria reduction in NS.

Next, the DAVID functional annotation cluster analysis tool, an internet-based gene function annotation software application, was used to determine which pathways or biological processes may be regulated by these DEGs in glomeruli 15. DAVID analysis of the 319 PAN-induced genes with high enrichment scores (i.e. P<0.05) revealed that most of these DEGs were involved in cell cycle/cell division/mitosis, nucleosome/chromatin-silencing/DNA-binding, and DNA-damage/repair functions (FIG. 1F). These findings suggested that PAN alters DNA dynamics, which may impact the overall survival of glomerular cells. In addition, gene sets associated with ATP binding, microtubule activity, P53 signaling, protein kinase activity/phosphorylation, ubiquitination, transcription factor complexes, and extracellular matrix (ECM) organization were also significantly altered (FIG. 1F). The precise molecular changes induced by PAN are not completely known, but these analyses suggest that glomerular DNA damage could potentially be the initiating event for PAN-induced proteinuria, partly in line with a previously published observation. Marshall et al., Kidney Int 70, 1962-1973 (2006).

An identical DAVID analysis of the 126 PAN-suppressed genes with high enrichment scores (i.e. P<0.05) revealed that most of these DEGs encompassed PDZ and SAM domains. PDZ domains are involved in anchoring membrane receptor proteins to cytoskeletal components. Lee, H.-J. & Zheng, J. J. Cell Communication and Signaling 8, 8 (2010). Proteins containing SAM domains exist in all subcellular locations, are involved in many different biological processes, bind to a variety of proteins, and have been shown to bind RNA. Green et al., Mol Cell 11, 1537-1548 (2003). In addition, gene sets associated with the activity of transcription factors, growth factors, and cytokines were also induced (i.e. ameliorated) by both MP and Pio treatments. Overall, the biological processes identified by these analyses suggest potentially important molecular pathways in glomeruli that are dysregulated during development of proteinuria in NS and ameliorated by treatments that effectively reduce the proteinuria.

### Drug-Nuclear Receptor DEG Interaction Network-Based Analysis of PAN-Induced and PAN-Suppressed DEGs Identified 20 Glomerular Genes-of-Interest (Method 1)

To attempt to identify novel drug targets using the PAN-induced and PAN-suppressed glomerular DEGs, the Ingenuity Pathway Analysis (IPA) was used to screen for DEGs that were similarly regulated by both proteinuria-reducing drugs (MP and Pio) and/or their known nuclear receptors (NR3C1 and PPARG, respectively). These analyses included using the Ingenuity Knowledge Base (IKB), a repository of expertly curated biological interactions and functional annotations, to search among the selected DEGs for simultaneous interactions between the nuclear receptors (NR3C1 and PPARG) and their respective agonists (methylprednisolone/dexamethasone and pioglitazone). Although methylprednisolone was used as the representative GC for our experiments, for the in-silico analyses we also included dexamethasone, another NR3C1 agonist, to compensate for the paucity of available IKB repository data on methylprednisolone. The results of this initial analysis identified an interaction network formed between these drugs, their nuclear receptors, and the glomerular DEGs (319 PAN-induced and 126 PAN-suppressed genes) resulted in the identification of 20 genes-of-interest (see bolded and enlarged gene names in FIG. 2A), whose protein products represent targets-of-interest. Since the proteins encoded by these genes are found in different cellular compartments, the inventors sought to identify any known interactions among the proteins that might reveal critical molecular pathways relevant to proteinuria reduction. To do this, the STRING database, a web resource of known and predicted protein-protein interactions, was used. Szklarczyk, D. et al., Nucleic Acids Res 47, D607-D613 (2019). Using k-means clustering, 4 distinct clusters of protein-protein interactions encoded by the 20 genes-of-interest were identified. These clusters are depicted by dotted bubbles in FIG. 2B, while the non-clustered/non-interactive nodes were removed from the interaction network. Consistent with the DVAID functional annotation analysis in FIG. 1E, the STRING analysis identified predicted protein interactions involved in 4 aspects of glomerular cell function: 1) Extracellular matrix homeostasis (Extracellular matrix/growth-factor activity), 2) DNA homeostasis (cell cycle and DNA-binding), 3) Lipid metabolism, and 4) Cytoskeletal organization. These interaction analyses complement the above DAVID analyses, and provide further support for dysregulation of a limited set of critical molecular pathways in glomeruli during development of proteinuria in NS and their amelioration by (mechanistically distinct) treatments that effectively reduce the proteinuria.

### Gene Set Enrichment Analyses Revealed Restoration of Dysregulated Glomerular Extracellular Matrix Genes as a Common Mechanism of Proteinuria Reduction (Method 2)

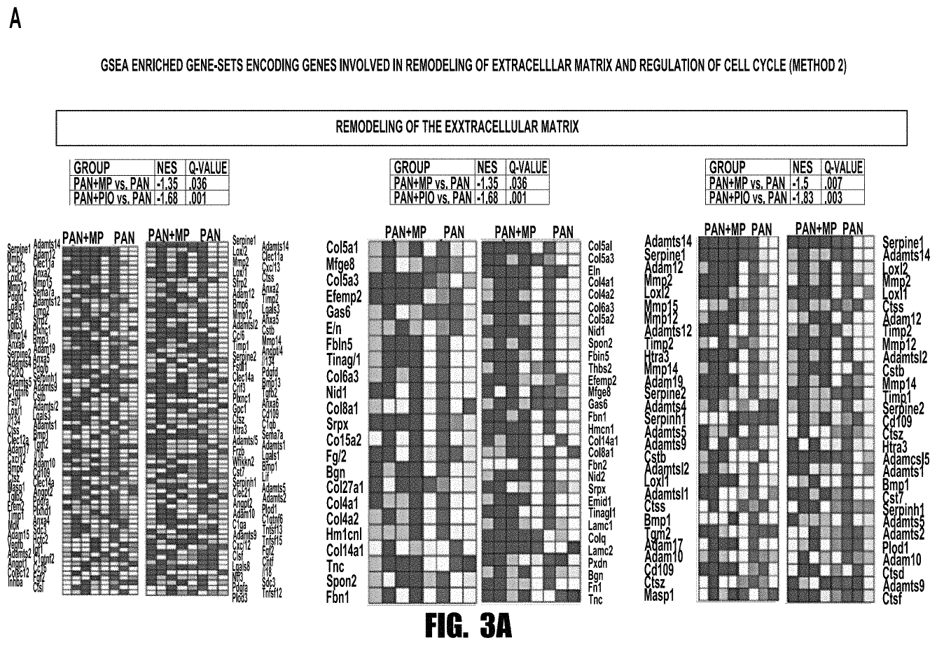

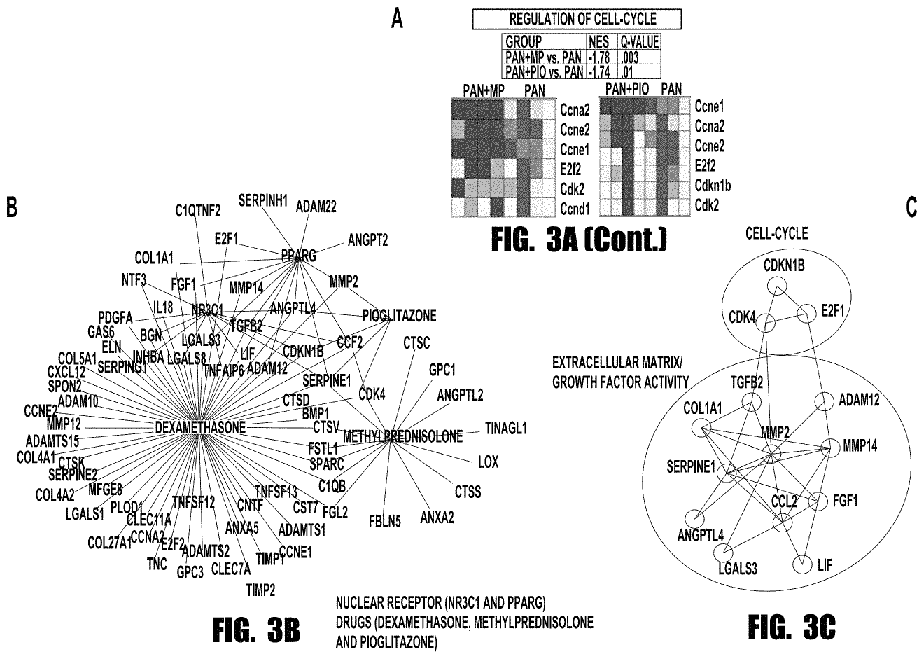

Gene set enrichment analysis (GSEA) is commonly employed for pathway analysis and functional annotation of gene sets identified by RNA-seq using a molecular signature database that currently includes 22,596 gene sets. Subramanian, A. et al., Proc Natl Acad Sci USA 102, 15545-15550 (2005). The inventors utilized GSEA as a second method to identify glomerular genes that are dysregulated during PAN-NS and ameliorated in response to effective (and mechanistically distinct) proteinuria reduction with both GC and Pio. The inventors evaluated 17,000 glomerular genes using a cutoff of log 2(Foldchange)≥2 to enforce significant stringency in the identification of enriched gene sets. In comparison to healthy control rats, PAN significantly (FDR<0.05) induced dysregulation of glomerular gene sets associated with both extracellular matrix (NABA_MATRISOME ASSOCIATED, NABA_ECM_REGULATORS, NABA_CORE_MATRISOME) and cyclins (SA_REG_CASCADE_OF_CYCLIN_EXPR), which were significantly ameliorated on GSEA analysis by both MP and Pio (see heat maps in FIG. 3A). These findings add further evidence supporting transcriptional dysregulation of glomerular ECM proteins and cyclins during PAN-NS, and amelioration of this dysregulation with effective proteinuria reduction.

Similar to the IPA analyses shown in FIG. 2A, these GSEA-derived enriched gene-sets were used in a separate attempt to identify novel drug targets for proteinuria reduction. For these studies the inventors used a Drug-Nuclear Receptor-DEGs Interaction Network-Based Analysis (part of IPA software) to analyze genes that were commonly regulated by both MP and Pio treatments (MP, dexamethasone, and Pio) and/or their nuclear receptors (NR3C1 and PPARG). This interaction network identified 14 genes-of-interest likely to be involved in NR3C1 and PPARG signaling processes (genes shown in bold in FIG. 3B). Further, these findings were extended to analyze protein-protein interactions among the proteins encoded by these 14 genes-of-interest using the STRING web-based application. Using k-means clustering, most of the targets clustered as an ECM-associated cluster, with matrix metalloproteinase 2 (MMP2) as a common interacting partner (see FIG. 3C).

Importantly, the genes-of-interest identified using the GSEA method (Method 2) overlapped significantly with the IPA method (Method 1), with most of the genes in common being involved with ECM remodeling (ADAM12, MMP14, LGALS3, SERPINE1). Therefore, both distinct informatic approaches to identify glomerular genes-of-interest that were common to both GC- and Pio-induced proteinuria reduction identified a potentially important role for amelioration of glomerular ECM protein dysregulation in NS.

Correlation of Genes-Of-Interest with Rat and Human Nephrotic Syndrome-Associated Glomerular Gene Expression

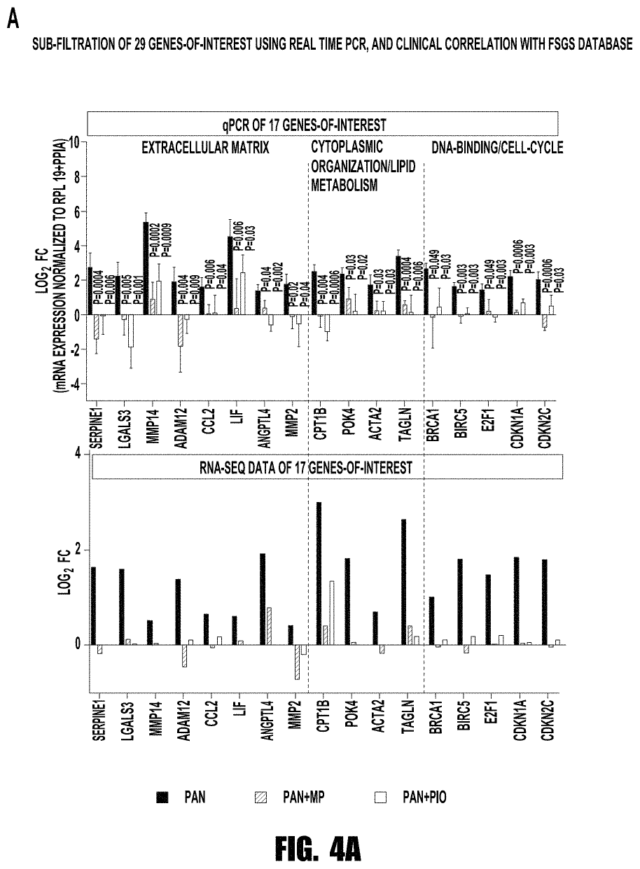

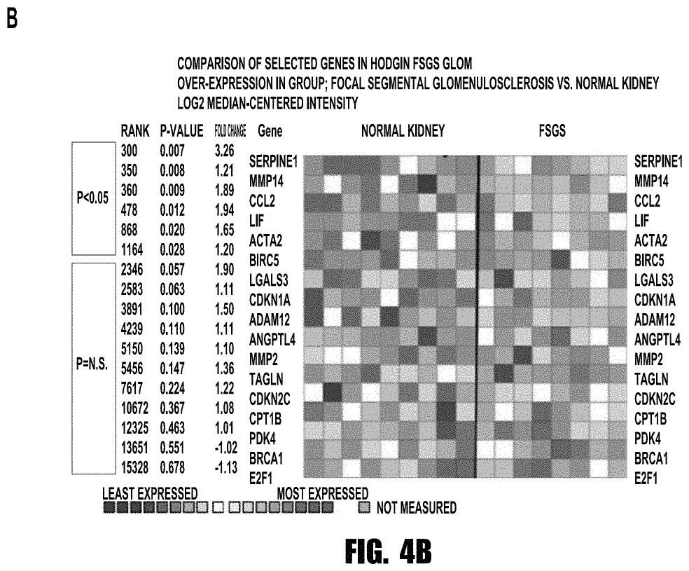

Despite known/reported close correlations between real-time PCR and RNAseq data (Wu, A. R. et al., Nat Methods 11, 41-46 (2014)), the inventors attempted to further validate the combined 29 RNAseq-derived targets-of-interest (Method 1+Method 2) using real-time PCR in the same cohort of rat samples used for the glomerular transcriptome preparations. As expected, the mRNA expression patterns across the various treatment groups appeared generally similar between the RNAseq and real-time PCR, validating the technical reproducibility of the findings. To further validate the findings the inventors evaluated only those genes that showed significantly different real-time PCR expression levels (P<0.05 by unpaired t-test) between PAN vs. PAN+MP and PAN+Pio, which narrowed our genes-of-interest from 29 down to 17 (see FIG. 4A).

The inventors then further validated these 17 genes-of-interest against clinically-derived human gene expression data from isolated glomeruli from FSGS patients in the Nephroseq (publicly available) database (see FIG. 4B). This analysis confirmed significantly altered glomerular expression of 6 of the 17 genes-of-interest in human FSGS (SERPINE1, MMP14, CCL2, LIF, ACTA2 and BIRC5), thus highlighting moderate concordance between NS-associated glomerular gene expression changes in rat vs. human NS.

Glucocorticoids and Pioglitazone Both Reverse PAN-Induced mRNA Changes in Podocyte—and Mesangial Cell-Specific, But Not Endothelial Cell-Specific, Gene Clusters

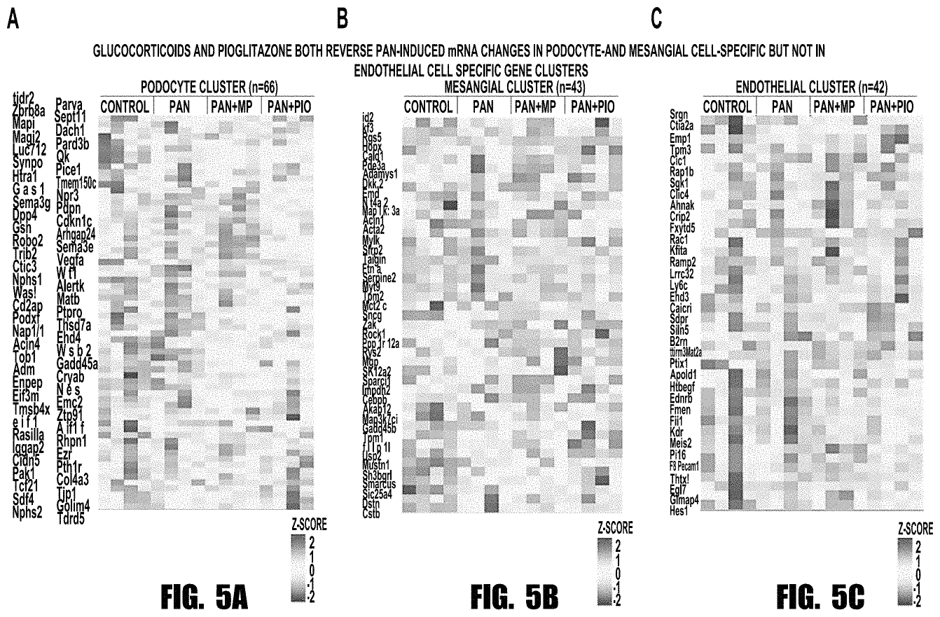

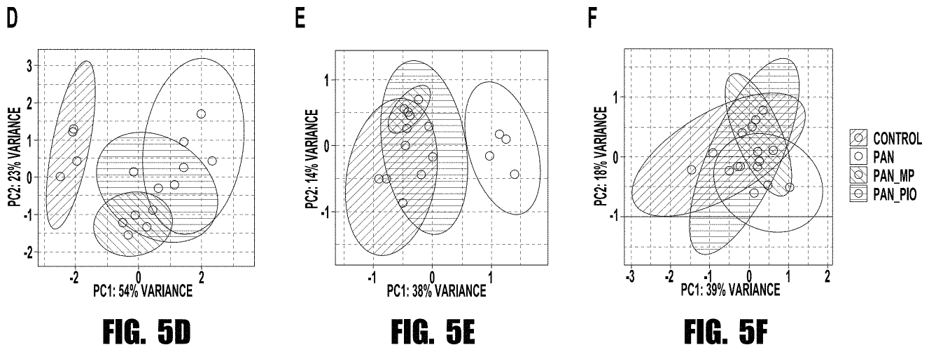

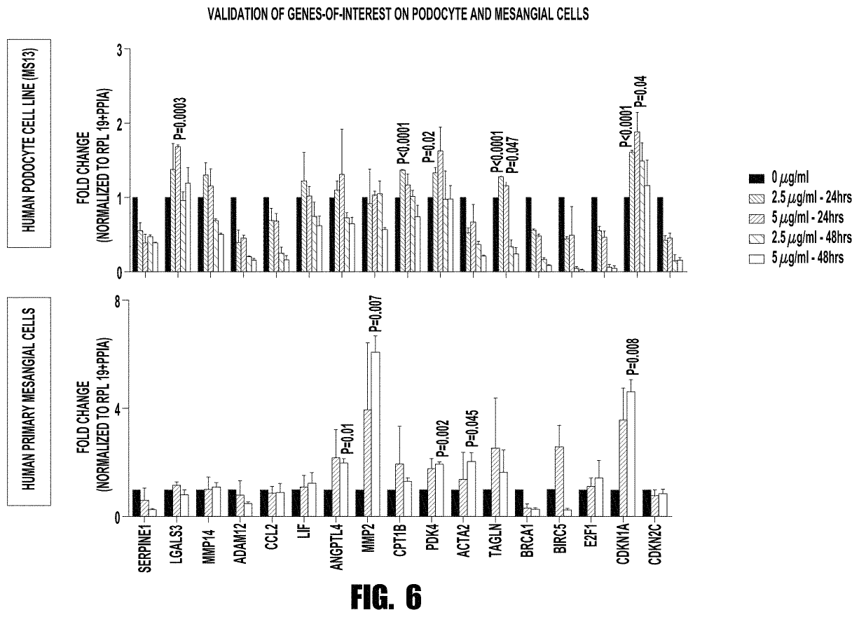

Normal glomerular function depends on coordinated signaling between three resident cell lineages: podocytes, endothelial cells, and mesangial cells. Merchant, M. L. et al. J Am Soc Nephrol 31, 1883-1904 (2020). While PAN-NS is a well-accepted model for the induction of glomerular proteinuria, its effects on mRNA dynamics in these three cell types has not been extensively studied. To enable the inventors to analyze glomerular cell-specific changes in gene expression during NS, they subdivided the glomerular transcriptomes into cell-specific subgroups using a published reference list of glomerular cell type-specific genes from healthy mouse glomeruli that was developed using single-cell RNA-seq to segregate podocyte-, endothelial cell-, and mesangial cell-specific genes among our glomerular transcriptomes. Karaiskos, N. et al., J Am Soc Nephrol 29, 2060-2068 (2018). For these analyses, the normalized mRNA counts in the glomerular transcriptomes varied from 0 to −400,000, so we cross-tabulated the genes with the previously published mouse glomerular single-cell RNA-seq data using a cut-off of ≥500 normalized mRNA counts to select genes for this cell-type specificity analysis. After applying this criterion, the inventors identified 66 podocyte-, 42 endothelial-, and 43 mesangial cell-specific genes. The heat maps shown in FIGS. 5A-C illustrate each of these three cell-type specific gene clusters, including comparisons of mRNA changes among the Control vs. PAN vs. PAN+MP vs. PAN+Pio treatment groups. Notably, the PAN-induced gene expression alterations were substantially reversed following either MP or Pio treatment in both podocytes and mesangial cells, as exemplified by the respective PCA plots (with 95% confidence interval color shading) derived from these heat maps (see FIGS. 5D and 5E).

In marked contrast to podocytes and mesangial cells, PAN treatment induced far fewer alterations in endothelial cells, and neither MP nor Pio treatment had notable effects on the transcriptional profiles of endothelial cell-specific genes, despite their effectiveness in reducing proteinuria in the rats (see FIG. 5F). In addition to these cell-specific findings, a combined PCA plot that included the above podocyte-, mesangial- and endothelial-specific genes also showed significant dysregulation of glomerular gene expression, with partial reversal of these changes (and significant overlap) following treatment with either MP or Pio. These findings suggest common gene products or pathways between MP and Pio in glomeruli that may have mediated the antiproteinuric effects in Rats, primarily via direct effects on podocytes and mesangial cells, or possibly via paracellular communication between podocytes and mesangial cells. Overall, these results suggest that focusing on podocyte and mesangial cell alterations in NS would be an auspicious approach to the development of future therapeutics for NS. In this context, the potential biological interpretations of these podocyte- and mesangial-specific mRNA dynamics were analyzed by incorporating the data into IPA-based function, disease, and toxicity analysis algorithms. This analysis indicated that compared to PAN-induced NS, treatment with either MP or Pio, both led to enhanced formation of filopodia, focal adhesions and microtubule dynamics, increased cell viability, decreased hyperplasia of mesangial cells, and decreased glomerular apoptosis.

### In-Vitro Validation of Genes-Of-Interest in Podocytes and Mesangial Cells

Since podocyte- and mesangial-specific gene expression changes on treatments (as shown in FIG. 5A-F), these sub-filtered 17 genes-of-interest were investigated directly in cultured human podocyte cell line and primary human mesangial cells by exposing these cells to PAN (human podocyte cell line: 2.5 and 5 μg/ml for 24 h and 48 h; human primary mesangial cells: 5 μg/ml for 24 h and 48 h; these time points and the conditions were chosen based on optimizations in the laboratory). Real-time PCR profiling of these 17 genes-of-interest in these cells revealed induction of expression of LGALS3 (also known as galectin-3) in podocyte specific manner (P=0.0003 vs. controls), whereas expression of MMP2 (P=0.007 vs. controls) and ACTA2 (P=0.045 vs. controls) were induced in mesangial cells specifically on exposure to PAN (FIG. 5B). However, genes (CPT1B, PDK4, ANGPTL4, TAGLN and CDKN1A) showed significant increase in expression in both cell-types which points towards the commonly regulated processes (lipid metabolism, cytoskeletal organization and cell-cycle) on exposure to PAN. Interestingly, SERPINE1, MMP14, CCL2, LIF, BIRC5 did not follow the expected pattern in either cell types could hint towards its source perhaps from a different cell type found in the glomerulus.

### DISCUSSION

The current study was designed to test the hypothesis that the similar antiproteinuric effects of Glucocorticoid and Pioglitazone are due to activation of common transcriptional profile. Therefore, glomerular transcriptome comparisons were made between PAN-induced NS and effective proteinuria reduction with GC (immunosuppressive) or Pio (non-immunosuppressive) treatment. Analyses included use of extensive in-silico approaches, glomerular cell type-specific gene set deconvolution, in vitro confirmatory studies in human podocytes and mesangial cells for lead target validation, and comparison of lead targets with the web-based Nephroseq human NS gene expression database. Collectively, these studies demonstrated that the similar antiproteinuric effects of GC and Pio in NS resulted from transcriptional regulation of both distinct and overlapping glomerular gene sets, and particularly, computationally identified reparation of dysregulated ECM associated genes by both treatments. In-depth analysis and cell validation studies pointed towards three genes LGALS3, MMP2 and ACTA2 that may play an important role in NS related pathophysiology. However, there is a need to mechanistically dissect the role LGALS3/MMP2/ACTA2 ECM regulatory pathways in a systematic fashion to prove that they modulate disease.

In the studies described in this example, the inventors attempted to define the molecular basis for these similar proteinuria-reducing effects by directly comparing transcriptomes from glomeruli isolated from rats with PAN-NS to those treated with PAN+MP and PAN+Pio. Subsequent PCA analyses of RNA-seq data from these transcriptomes revealed generally similar therapeutic transcriptional activation patterns between the PAN+GC and PAN+Pio groups, with both treatment groups demonstrating substantial reversal of the PAN-induced changes in transcriptional clusters associated with NS. Of note, many of DEGs in the PAN+Pio group overlapped extensively with those in the PAN+GC group, pointing towards drugs' proteinuria-reducing effects could potentially be associated with similar alterations in within glomeruli. While it was expected that GC treatment would correct the expression of many glomerular genes dysregulated by PAN-induced NS, since prednisone (a widely used GC in clinical practice) is considered a front-line medication for NS (Ponticelli, C. & Passerini, P. Kidney Int 46, 595-604 (1994)), it was of particular interest that Pio demonstrated the similar extent of reversibility in glomerular gene expression. Indeed, a Venn diagram analysis of filtered DEGs revealed a total of 445 gene sets (319 PAN-induced+126 PAN-suppressed) that overlapped between MP and Pio treatments, contributing to the similar antiproteinuric effect observed between the two treatments. DAVID functional annotation tool used to compute biological pathways from the list of genes (Huang da et al., Nat Protoc 4, 44-57 (2009)) revealed a modest list of potentially targetable biological pathways and processes. While PAN treatment induced or suppressed several processes, the manifestation of these processes resulted in abnormal ECM remodeling—a prominent feature of many glomerular diseases. Hobeika et al., Kidney Int 91, 501-511 (2017). To elaborate on this, two analysis methods (Method 1 and Method 2, respectively) were utilized to critically narrow down the lead target discovery to 29 auspicious genes-of-interest. Method 1 employed manual based approach of filtering DEGs according to the statistical criteria applied, that identified revealed 20 genes-of-interest—commonly regulated by the nuclear receptors NR3C1 and PPARG, and their respective agonists (GC and Pio). These 20 genes-of-interest resulted in four interactive clusters of the proteins encoded using IPA software: 1) DNA-binding, 2) Extracellular matrix (ECM), 3) Lipid metabolism, and 4) Cytoskeletal organization (see FIG. 2B). Of note, the focal point of these interactive clusters was the ECM and ECM-like network.

Interestingly, method 2 employed more global and unbiased approach of screening pathways/biological processes: Gene set enrichment analysis (GSEA, which is a very powerful method for the global analysis of transcriptomic data, revealed amelioration of PAN-induced changes by GC and Pio in gene-sets encoding matrisome, ECM, ECM-related, cyclin, and core matrisome proteins, on which the interaction network applied revealed 14 gene-of-interest-commonly regulated by the nuclear receptors NR3C1 and PPARG, and their respective agonists (GC and Pio). However, the overlap between 20 genes-of-interest from method 1 and 14 genes-of-interest from method 2 resulted in 29 lead targets for further exploration. Of 29 lead targets, 13 are associated ECM affiliated gene-sets, 10 are associated with DNA binding/Cell-Cycle gene-sets, rest of the genes are associated with lipid metabolism, cytoskeletal rearrangement and mitochondrial. Real PCR time evaluation of 29 gene-of-interest revealed a similar expression patterns as from RNA-seq data, demonstrating a technical reproducibility of the findings, however, on setting a p<0.05 cut-off between PAN vs. PAN+GC and PAN vs. PAN+Pio as a sub-filtration criterion, 17 genes-of-interest were selected for further evaluation. On identifying their role in biological processes using Gene Ontology—Biological Processes analysis, these 17 genes-of-interest were categorized majorly into ECM regulation followed by DNA-binding/Cell-Cycle process and then cytoplasmic rearrangement and lipid metabolism. Overall, the combined analysis shed light on the understanding of ECM regulation as an effective approach to discover targeted therapies for NS. The genes of interest from Method 1 and Method 2 are shown in Tables 1 & 2.

TABLE 1
Genes of Interest from Method 1 with their respective
localization and biological activity

| Method 1 | Symbol | Entrez Gene Name | Location | Type |
|---|---|---|---|---|
| 1 | ACTA2 | Actin alpha 2, smooth muscle | Cytoplasm | other |
| 2 | ADAM12 | ADAM metallopeptidase domain 12 | Plasma | peptidase |
| | | | Membrane | |
| 3 | AIFM3 | Apoptosis inducing factor | Cytoplasm | enzyme |
| | | mitochondrial associated 3 | | |
| 4 | BIRC5 | Baculoviral IAP repeat containing 5 | Cytoplasm | other |
| 5 | BRCA1 | BRCA1 DNA repair associated | Nucleus | Transcription |
| | | | | regulator |
| 6 | CDKN1A | Cyclin dependent kinase inhibitor 1A | Nucleus | kinase |
| 7 | CDKN2C | Cyclin dependent kinase inhibitor 2C | Nucleus | Transcription |
| | | | | regulator |
| 8 | CPT1B | Carnitine palmitoyl transferase 1B | Cytoplasm | enzyme |
| 9 | E2F1 | E2F transcription factor 1 | Nucleus | Transcription |
| | | | | regulator |
| 10 | FOSB | FosB proto-oncogene, AP-1 | Nucleus | Transcription |
| | | transcription factor subunit | | regulator |
| 11 | FOSL1 | FOS like 1, AP-1 transcription factor | Nucleus | Transcription |
| | | subunit | | regulator |
| 12 | IGFBP5 | Insulin like growth factor binding | Extracellular | other |
| | | protein 5 | Space | |
| 13 | LBALS3 | Galactin 3 | Extracellular | other |
| | | | Space | |
| 14 | LPL | Lipoprotein lipase | Cytoplasm | enzyme |
| 15 | MMP14 | Matrix metallopeptidase 14 | Extracellular | peptidase |
| | | | Space | |
| 16 | PDK4 | Pyruvate dehydrogenase kinase 4 | Cytoplasm | kinase |
| 17 | POU2F1 | POU class 2 homeobox 1 | Nucleus | Transcription |
| | | | | regulator |
| 18 | TAGLN | transgelin | Cytoplasm | other |
| 19 | SERPINE1 | Serpine family E member 1 | Extracellular | Peptidase |
| | | | Space | regulator |
| 20 | VEGFA | Vascular Endothelial Growth Factor | Extracellular | Growth |
| | | | Space | factor |

TABLE 2
Genes of Interest from Method 1 with their respective
localization and biological activity

| Method 2 | Symbol | Entrez Gene Name | Location | Type |
|---|---|---|---|---|
| 1 | COL1A1 | Collagen type 1 alpha 1 chain | Extracellular | other |
| | | | Space | |
| 2 | FGF1 | Fibroblast growth factor 1 | Extracellular | Growth |
| | | | Space | factor |
| 3 | LGAL53 | Galactin 3 | Extracellular | other |
| | | | Space | |
| 4 | TGFB2 | Transforming growth factor beta 2 | Extracellular | Growth |
| | | | Space | factor |
| 5 | ANGPTL4 | Angiopoietin like 4 | Extracellular | other |
| | | | Space | |
| 6 | MMP14 | Matrix metallopeptidase 14 | Extracellular | peptidase |
| | | | Space | |
| 7 | E2F1 | E2F transcription factor 1 | Nucleus | Transcription |
| | | | | regulator |
| 8 | LIF | LIF interleukin 6 family cytokine | Extracellular | cytokine |
| | | | Space | |
| 9 | MMP2 | Matrix metallopeptidase 2 | Extracellular | peptidase |
| | | | Space | |
| 10 | ADAM12 | ADAM metallopeptidase domain 12 | Plasma | peptidase |
| | | | Membrane | |
| 11 | CCL2 | Chemokine (C-C motif) ligan 2 | Extracellular | cytokine |
| | | | Space | |
| 12 | CDK4 | Cyclin dependent kinase 4 | Nucleus | kinase |
| 13 | CDKN18 | Cyclin dependent kinase inhibitor 1B | Nucleus | kinase |
| 14 | SERPINE1 | Serpine family E member 1 | Extracellular | Peptidase |
| | | | Space | regulator |

For the clinical significance, the inventors validated the 17 genes-of-interest from their study with the glomeruli transcriptomic data from FSGS patients curated in a Nephroseq database. Although, some of the genes did not statistically correlated with our genes-of-interest, which could pin-point to the limitations posed by FFPE sample processing, indicating the need for effective technologies such as 10× spatial transcriptomics. Nevertheless, they proceeded with validation of these genes-of-interest in in-vitro culture systems. In order to address which particular cell to investigate, the computational deconvolution to glomerular cell types was performed. As it is understood that within glomeruli, multidirectional cross talk among resident podocytes, mesangial cells, and endothelial cells occurs. Kitching, A. R. & Hutton, H. L., Clin J Am Soc Nephrol 11 (2016). Using published data from single cell RNA sequencing of each cell type of mouse glomeruli to segregate the genes in their transcriptomes (Karaiskos, N. et al. J Am Soc Nephrol 29, 2060-2068 (2018)), they identified distinctly different patterns of gene alterations in podocytes, mesangial cells, and endothelial cells in the PAN vs. PAN+MP vs. PAN+Pio treatment groups. The gene dysregulation with induction of NS, and reversal following GC or Pio treatment was seen in podocytes and mesangial cells, and only modest changes were seen in endothelial cells, therefore the source of targets-of-interest were evaluated in PAN exposed cultured human podocytes and human primary mesangial cells. Of all targets, expression of LGALS3 was exclusively enhanced post-PAN treatment in podocytes. LGALS3 which encodes the Galectin-3 protein has been shown to regulate cell growth, differentiation, and inflammation. LGALS3 has also been shown to regulate cell adhesion and migration by regulating expression of integrin a6131 and a3131 leading to actin cytoskeletal organization in glioma (Debray, C. et al., Biochem Biophys Res Commun 325, 1393-1398 (2004)), which actually makes it a relevant target as it is understood that the importance of these integrins in podocyte adhesion to glomerular basement membrane. Clinically, increased protein levels of LGALS3 have been shown to be present in plasma of patients with increased risks of rapid renal function decline (Rebholz, C. M. et al., Kidney Int 93, 252-259 (2018)), and is also present in human glomerular ECM proteome of collapsing FSGS patients. Merchant, M. L. et al., J Am Soc Nephrol 31, 1883-1904 (2020). In mesangial cells, MMP2 and ACTA2 expression was markedly upregulated on exposure to PAN. Matrix Metalloproteinase 2 or MMP2 is a gelatinase capable of degrading glomerular basement membrane and α-smooth muscle actin (ACTA2) has been shown to control contractile processes in high glucose induced mesangial cells. Han et al., Exp Ther Med 14, 181-186 (2017). Interestingly, in the protein-protein interaction analysis, MMP2 has been shown to be interacting with other ECM associated protein interacting partners such as ADAM12, MMP14, FGF1, CCL2, SERPINE1, COL1A1 AND TGFB2 suggesting MMP2 as a central player in ECM remodeling. Also, MMP2 expression was higher in glomeruli isolated from steroid-resistant nephrotic syndrome patients. This clearly indicates that further exploration of LGLAS3/MMP2/ACTA2 axis can lead us the development of future proteinuria-reducing treatments for NS.

In summary, the inventors discovered relevant genes-of-interest that may play an important role in pathobiology of NS. These genes were discovered on the basis in-silico comparison and analysis of transcriptomics data derived from steroidal and non-steroidal treatment and functional validation of lead targets. The novelty lies in the utility of FDA-approved drug pioglitazone as a nonsteroidal counterpart to steroids and in approach of analyzing the rat glomerular transcriptomics data by streamlining and focusing on the overlapping gene-sets between GC and Pio, and further testing these selected lead targets in immortalized human podocytes and primary human mesangial cells. Importantly, most of these selected lead targets correlated clinically with FSGS patient cohort dataset from Nephroseq. Overall, this study sets a platform for non-immunosuppressive treatment for NS but further evaluation for mechanistic involvement of LGALS3/MMP2/ACTA2 in the pathobiology of NS will be required for development of targeted therapies.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of identifying one or more genomic regions associated with kidney disease and/or its treatment, comprising:
administering a glucocorticoid to a first group of subjects;
administering a thiazolidinedione to a second group of subjects; and
identifying a plurality of genomic regions affected in the first group and the second group,
wherein the subjects have a kidney disease or are animal models of a kidney disease.

2. The method of claim 1, wherein the kidney disease is nephrotic syndrome and the subjects have nephrotic syndrome or are animal models of nephrotic syndrome.

3. The method of claim 1, wherein the glucocorticoid is methylprednisolone or dexamethasone.

4. The method of claim 1, wherein the thiazolidinedione is pioglitazone.

5. The method of claim 1, wherein the genomic regions are identified using RNA sequencing.

6. The method of claim 5, wherein the RNA sequencing is performed on RNA isolated from the kidney glomeruli of the subjects.

7. The method of claim 1, wherein the genomic regions affected are identified as being either upregulated or downregulated compared to untreated disease.

8. The method of claim 1, wherein the genomic regions that are upregulated or downregulated in both the first and second groups are identified.

9. The method of claim 1, further comprising analysis of the target-drug interaction network using Ingenuity pathway analysis of the identified genomic regions.

10. The method of claim 9, wherein ingenuity pathway analysis characterizes the genomic regions as being involved in the genes selected from the group consisting of genes relating to the extracellular matrix, core-matrisome, cell-cycle, DNA damage-repair, lipid metabolism, growth factors, cytokine activity, cell proliferation, and cell membrane glycoprotein levels.

11. The method of claim 1, further comprising characterizing the genomic regions identified using functional annotation cluster analysis.

12. The method of claim 11, wherein functional annotation cluster analysis characterizes the genomic regions as being involved in the genes selected from the group consisting of genes relating to the extracellular matrix, core-matrisome, cell-cycle, DNA damage-repair, lipid metabolism, growth factors, cytokine activity, cell proliferation, and cell membrane glycoprotein levels.

13. The method of claim 11, wherein 20 to 40 genomic regions are identified as genomic regions of interest

14. The method of claim 1, wherein the genomic regions are selected from the group of genes associated with functions consisting of cartinine palmitotransferase 1B (CPT1B), transgelin (TAGLN), pyruvate dehydrogenase kinase 4 (PDK4), cyclic dependent kinase inhibitor 1B (CDKN1B), cyclic dependent kinase inhibitor 1A (CDKN1A), cyclin-dependent kinase inhibitor 2C (CDKN2C), baculoviral IAP repeat containing 5 (BIRC5), serpin family E member 1 (SERPINE1), E2F transcription factor 1 (E2F1), ADAM metallopeptidase domain 12 (ADAM12), BRCA1 DNA repair associated (BRCA1), FosB proto-oncogene (FOSB), AP-1 transcription factor subunit, fos-like antigen 1 (FOSL1), actin gamma 2 (ACTA2), lipoprotein lipase (LPL), matrix metalloproteinasel4 (MMP14), matrix metalloproteinase 2 (MMP2), apoptosis inducing factor mitochondria associated 3 (AIFM3), vascular endothelial growth factor A (VEGFA), and insulin-like growth factor binding protein 5 (IGFBP5), collagen, type 1, alpha 1 (COL1A1), lectin galactoside-binding soluble 3 (LGALS3) angiopoietin-like 4 (ANGPTL4), POU class2 homeobox 1 (POU2F1), leukemia inhibitory factor (LIF), fibroblast growth factor 1 (FGF1), chemokine (c-c motif) ligand 2 (CCL2), cyclin-dependent kinase 4 (CDK4) and transforming growth factor b2 (TGFB2)

15. The method of claim 1, wherein each of the genomic regions identified is characterized as being podocyte-specific, endothelial cell-specific, or mesangial cell-specific.

16. A method of identifying a drug for treatment of nephrotic syndrome, comprising administering a glucocorticoid to a first group of subjects; administering thiazolidinedione to a second group of subjects; identifying a plurality of genomic regions affected in the first group and the second group, and identifying a drug for treatment of nephrotic syndrome if it is known to affect a genomic region affected in the first group and/or the second group of subjects;
wherein the subjects have a kidney disease or are animal models of a kidney disease.
